(19) United States
(12) Patent Application Publication
(10) Pub. No.: US 2026/0306354 A1
(43) Pub. Date: Oct. 8, 2026

(54) THERAPEUTIC DEVICE WITH A FUNCTIONAL HEAD

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Tang Yuanzhou, Shenzhen (CN); Feng Jianxin, Shenzhen (CN)

(73) Assignee: SHENZHEN NUON MEDICAL EQUIPMENT CO., LTD., Shenzhen (CN)

(21) Appl. No.: 19/392,434

(22) Filed: Nov. 18, 2025

(30) Foreign Application Priority Data
Nov. 22, 2024 (CN) 2024228561059

(51) Int. Cl. A61H 23/02; A61N 5/06

(52) U.S. Cl. CPC A61H 23/02; A61N 5/0616; A61H 2201/0107; A61H 2201/0153; A61H 2201/10; A61H 2201/1671; A61N 2005/0644

(57) ABSTRACT

Embodiments of the present invention disclose a multifunctional therapeutic device comprising a handle and a functional head with a pair of elastically deformable connecting arms defining a massage space. Each connecting arm is provided with a rotatable massage element, and the head incorporates a phototherapy lamp for emitting light between the arms to promote skin rejuvenation. The device further includes stimulation elements such as a vibration motor, heating or cooling component, or microcurrent electrode, enabling combined therapeutic effects. A knob-controlled power mechanism in the handle allows selective activation of the internal circuit connecting the battery and main control board. The device may also feature a liquid channel and a detachable liquid container for dispensing skincare formulations through an outlet positioned between the arms. The invention provides integrated massage, phototherapy, and topical therapy in a compact, ergonomic handheld design.

### TECHNICAL FIELD

The present invention relates to the field of therapeutic and cosmetic care devices, and more particularly to a multifunctional therapeutic device configured to deliver various forms of treatment, including phototherapy, heat therapy, cooling therapy, vibration therapy, microcurrent therapy, and liquid application. The invention specifically pertains to a handheld massaging apparatus having a functional head and a handle integrated with electronic, optical, and mechanical components for providing skin stimulation, relaxation, and rejuvenation through light, vibration, microcurrent, etc., and topical application in a single compact structure.

### BACKGROUND ART

Massage and skincare devices are commonly used to promote relaxation, stimulate circulation, and improve the appearance and condition of the skin. Conventional devices of this type often rely on basic mechanical arrangements, such as fixed U-shaped heads or manually operated rollers, which provide only limited therapeutic engagement. Although some powered devices incorporate vibration or heating functions, such features are generally offered in isolated form and are not well adapted for use on the curved and uneven contours of the human face, including areas such as the nose bridge, cheekbones, jawline, chin, eyebrows, or forehead.

Phototherapy instruments are also known and are used to expose the skin to specific wavelengths of light for rejuvenation or inflammation control. However, such products are typically bulky, stationary, and separate from massage tools, thereby requiring users to operate multiple devices to achieve comprehensive treatment. Similarly, existing beauty devices that allow cosmetic liquid application tend to be single-purpose tools, offering either dispensing or massaging functionality, but not a coordinated combination of both. Rigid or non-adjustable treatment heads further reduce the ability of known devices to uniformly contact and treat different facial profiles, which can lead to uneven pressure distribution and reduced overall effectiveness.

Accordingly, there remains a need for a compact, handheld device capable of delivering multiple therapeutic functions, including mechanical stimulation, phototherapy, vibration, temperature-based treatment, and optional liquid dispensing, in an integrated manner. There is also a need for a treatment head that conforms naturally to curved body surfaces while providing stable structural support for embedded stimulation components. Furthermore, an improved and reliable power-control mechanism is desirable to enable users to readily activate internal circuitry in a controlled and intuitive manner.

The present invention addresses these and other shortcomings by providing a multifunctional therapeutic device having a flexible or elastically adjustable functional head, one or more integrated stimulation elements, and a handle containing the internal power and control components. In certain embodiments, the device additionally incorporates a liquid supply channel and an optional detachable container for delivering skincare or therapeutic liquids during treatment. The coordinated configuration of these structural features enables combined phototherapy, vibration massage, thermal therapy, and liquid application to be performed simultaneously or selectively within a single portable device.

### OBJECTS OF THE INVENTION

Some of the objects of the invention are as follows:

An object of the present invention is to provide a therapeutic device that integrates phototherapy, vibration massage, magneto-therapy, temperature-based therapy, and liquid application in a single handheld device, thereby delivering comprehensive skincare and therapeutic benefits.

Another object of the invention is to provide a functional head with dual connecting arms defining a massage space, wherein at least one connecting arm is elastically deformable, allowing the device to adapt to different facial contours and provide uniform, comfortable massage pressure on various regions of the human body.

A further object of the invention is to provide a flexibly covered functional head having soft contact surfaces, such as a flexible cover comprising first and second covering portions, to enhance user comfort, prevent skin irritation, and ensure gentle yet effective massage even on sensitive areas such as the nose, cheekbones, eyebrows, jawline, chin, wrists, arms, legs, and forehead etc.

Yet another object of the invention is to provide a functional head incorporating rotatable massage elements, such as roller balls, that can roll smoothly over the skin, reducing friction while improving blood circulation and absorption of skincare formulations.

An additional object of the invention is to provide a phototherapy lamp integrated within the functional head, positioned between the connecting arms to emit therapeutic light directly into the massage space, enabling simultaneous light therapy and mechanical stimulation for enhanced treatment results.

Another object of the invention is to provide a temperature therapy component, such as a cooling or heating element, made of materials including rock, ceramic, or metal, allowing the device to deliver temperature-based therapy in conjunction with massage or light treatment.

A further object of the invention is to provide a knob-controlled power mechanism within the handle, enabling the user to switch the circuit ON or OFF through simple mechanical rotation, which establishes or disconnects electrical contact between the battery and the main control board, ensuring safe and convenient power operation.

Yet another object of the invention is to provide an integrated vibration motor configured to generate therapeutic vibration transmitted through the functional head, thereby enhancing tissue stimulation, muscle relaxation, and overall massage effectiveness.

An additional object of the invention is to provide a liquid application system comprising a liquid channel extending through the handle and a liquid outlet port disposed between the connecting arms, allowing for controlled dispensing of skincare or therapeutic liquids during massage.

Another object of the invention is to provide an optional detachable liquid storage container attachable to the handle or the base portion, allowing users to refill or replace skincare liquids conveniently and maintain hygiene.

A further object of the invention is to provide an ergonomic handle configured for comfortable gripping and operation, with an obtuse-angled connection between the functional head and the handle to facilitate better contact with curved regions of a human body.

Yet another object of the invention is to provide a modular head assembly that includes locking notches, plug-in parts, and flexible embedding structures, ensuring a stable connection between components and enhancing manufacturing efficiency and durability.

A further object of the invention is to provide a rechargeable power system integrated within the handle, including the battery, charging component, and main control board, allowing for cordless operation and efficient power management.

Another object of the invention is to provide a multifunctional and hygienic therapeutic device that overcomes the drawbacks of traditional massagers and beauty tools that offer limited treatment modes, require multiple devices for different therapies, or lack ergonomic adaptability and ease of cleaning.

### SUMMARY OF THE INVENTION

According to a first aspect of the present invention, a therapeutic device is provided. The therapeutic device comprises a handle; and a functional head disposed at one end of the handle, the functional head comprises a pair of connecting arms spaced apart to define a massage space; a phototherapy element disposed in the massage space for emitting therapeutic light between the connecting arms; at least one stimulation element disposed in the functional head; and wherein the stimulation element is configured to deliver one or more therapies to a user.

In one embodiment of the invention, the connecting arms are elastically deformable to vary the width of the massage space.

In one embodiment of the invention, each connecting arm includes a rotatable massage element configured to provide a massage therapy to the user.

In one embodiment of the invention, at least one stimulation element comprises one of a heating element, a cooling element, a Peltier component, a magnetotherapy element, a vibration element, or a micro-current element.

In one embodiment of the invention, the functional head further comprises a flexible cover enclosing at least a part of the connecting arms.

In one embodiment of the invention, the functional head further comprises support and a connecting seat, the support connecting the pair of connecting arms, and the connecting seat being fixed to the handle.

In one embodiment of the invention, the support includes a plug-in part, and the connecting seat includes a plug hole, the plug-in part being inserted into the plug hole to couple the support and the connecting seat.

In one embodiment of the invention, an end of the connecting seat includes a notch penetrating the inner and outer sides of the connecting seat, and a first covering portion of the flexible cover is embedded within the notch.

In one embodiment of the invention, the connecting seat includes a plurality of locking holes, and a second covering portion of the flexible cover is embedded within the locking holes to secure the functional head.

In one embodiment of the invention, the functional head is inclined relative to the handle at an obtuse angle to facilitate contact with curved facial regions, including nose, jawline, cheekbones, and forehead.

According to a second aspect of the present invention, a therapeutic device is provided comprising a handle; a functional head disposed at one end of the handle, the functional head having a liquid outlet port positioned between a pair of connecting arms; one or more stimulation elements disposed in the functional head for providing one or therapies to a user; a liquid channel extending through the handle and communicating with the liquid outlet port; and a liquid storage container detachably coupled to the handle and in fluid communication with the liquid channel, wherein the liquid storage container is configured to deliver cosmetic or therapeutic liquid to the functional head.

In one embodiment of the invention, a roller ball is disposed at the liquid outlet port and configured to dispense liquid upon rolling contact with the skin of the user.

In one embodiment of the invention, the handle includes a mounting portion for attachment to the liquid storage container.

In one embodiment of the invention, the liquid channel extends from an inlet at the mounting portion to the liquid outlet port of the functional head.

In one embodiment of the invention, the liquid storage container is removably attachable to enable replenishment or cleaning.

In one embodiment of the invention, one or more stimulation elements are selected from a phototherapy lamp or vibration module to provide combined liquid application and therapy.

According to a third aspect of the invention, a therapeutic device with a knob-controlled power mechanism is provided. The therapeutic device comprising: a handle configured to house a battery and a main control board; a functional head disposed at one end of the handle and including at least one stimulation element electrically connected to the main control board; and a knob-controlled power mechanism comprising a knob, disposed at a base of the handle, wherein rotation of the knob retracts an insulating element to allow a terminal of the battery to contact a conductive element, thereby completing an electrical circuit through the conductive element, a spring, and the main control board to power the stimulation element.

In one embodiment, the knob is manually rotatable between ON and OFF positions, providing a simple and intuitive user interface for power control. The spring biases the insulating element toward the battery terminal in the OFF position to prevent electrical contact and ensure user safety. Upon rotation to the ON position, the circuit is completed, activating the phototherapy, vibration, or heating functions as regulated by the main control board.

In one embodiment, the main control board is configured to regulate and coordinate the operation of multiple stimulation elements, such as controlling the intensity, mode, and duration of the phototherapy lamp, vibration motor, or temperature module, in response to user input or pre-set operating modes. The power and control system may also be configured for wired or wireless charging, providing flexibility and ease of use.

The invention thus provides a multifunctional handheld therapeutic device that integrates multiple treatment modalities, including massage, phototherapy, vibration, temperature therapy, and liquid application. The flexible and ergonomic design allows it to adapt to various body contours, ensuring precise and comfortable contact. The combination of a knob-based power system, detachable liquid mechanism, and modular head structure provides convenience, safety, and a superior user experience.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “handheld therapy device” refers to any device configured to emit therapeutic light for skin treatment, pain relief, or wellness applications.

In the context of the specification, the term “stimulation element” refers broadly to any component, module, or structure configured to apply a therapeutic or cosmetic stimulus to a user's skin or tissue. Stimulation elements may include, but are not limited to, phototherapy elements, massage elements, microcurrent electrodes, ultrasonic transducers, heating elements, cooling elements, or combinations thereof.

In the context of the specification, the term “phototherapy element” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the specification, the term “massage element” refers to any component adapted to apply mechanical stimulation to the skin, including rotating rollers, kneading members, vibrating members, or reciprocating structures. The massage element may be fixed, detachable, or mounted for rotation or vibration relative to the housing.

In the context of the specification, the term “microcurrent element” refers to any electrode or conductive structure configured to deliver a controlled electrical signal to the user's skin. Such elements may include paired electrodes, conductive surfaces, or pads connected to a circuit board for generating microcurrent, galvanic current, or equivalent electrical therapy.

In the context of the specification, the term “housing” is intended to cover any casing, enclosure, or structural body that contains or supports components of the device. The housing may include a handle portion, head portion, or other segments, and may be made from polymeric, metallic, composite, or other suitable materials.

In the context of the specification, the terms “head” or “phototherapy head” refer to a portion of the device coupled to the housing and configured to emit light toward the skin. The head may include one or more light-transmitting surfaces, optical lenses, or diffusers, and may also support electrodes or other stimulation elements.

In the context of the specification, the term “circuit board” encompasses any printed circuit board (PCB), flexible circuit, or equivalent substrate that supports and electrically connects components of the device, including power supplies, control chips, drivers, or stimulation elements.

In the context of the specification, the term “user” or “subject” is intended to broadly cover humans, animals, or other recipients of the treatment, unless otherwise specifically limited.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557B 2 , titled “Roll-to-roll fabricated light sheet and encapsulated semiconductor circuit devices”, which is included herein in its entirety by reference.

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

The red light (approximately 630-660 nm) penetrates deeply into the scalp to stimulate blood circulation and enhance hair follicle activity, thus promoting hair growth and repair. Blue light (around 415-470 nm) exhibits antibacterial properties and is effective in treating scalp acne and reducing inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1A illustrates a perspective view of a functional head of a therapeutic device, in accordance with an embodiment of the present invention.

FIG. 1B illustrates a perspective view of another configuration of a functional head of the therapeutic device, in accordance with an embodiment of the present invention

FIG. 2 illustrates an exploded view of the functional head, in accordance with an embodiment of the present invention.

FIG. 3 illustrates a front view of the therapeutic device, in accordance with an embodiment of the present invention.

FIG. 4 illustrates a cross-sectional view of the therapeutic device, in accordance with an embodiment of the present invention.

FIG. 5 illustrates a cross-sectional view of another configuration of the therapeutic device with an integrated liquid storage container, in accordance with an embodiment of the present invention.

FIG. 6 illustrates a perspective view of another configuration of the therapeutic device, in accordance with an embodiment of the present invention.

FIG. 7 illustrates a cross-sectional view of the therapeutic device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a therapeutic device designed to provide comprehensive skincare and effective therapy to the nose region, targeting areas such as the nasal bridge, sides, and wings of the nose, through the integration of stimulation elements providing phototherapy, vibration massage, magneto-therapy, temperature therapy, microcurrent therapy, and liquid application, etc. The therapeutic device primarily consists of a handle and a functional head, wherein the functional head includes a pair of connecting arms defining a massage space and equipped with rotatable massage elements for smooth contact with the skin. The functional head further houses stimulation elements, such as a phototherapy lamp, vibration motor, and optional heating, cooling, or microcurrent elements, enabling multiple therapeutic effects in one unit. The handle incorporates a power supply system, a main control board, and a knob-operated power mechanism for efficient operation. Furthermore, a liquid channel and detachable container are provided to deliver skincare liquids during treatment, creating a compact, ergonomic, and versatile therapeutic device.

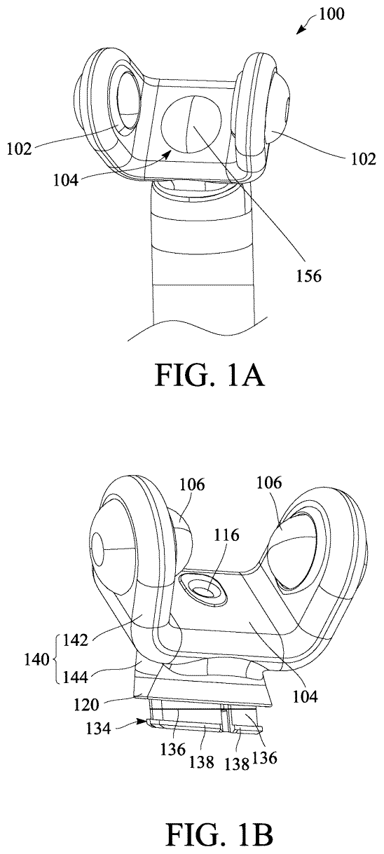

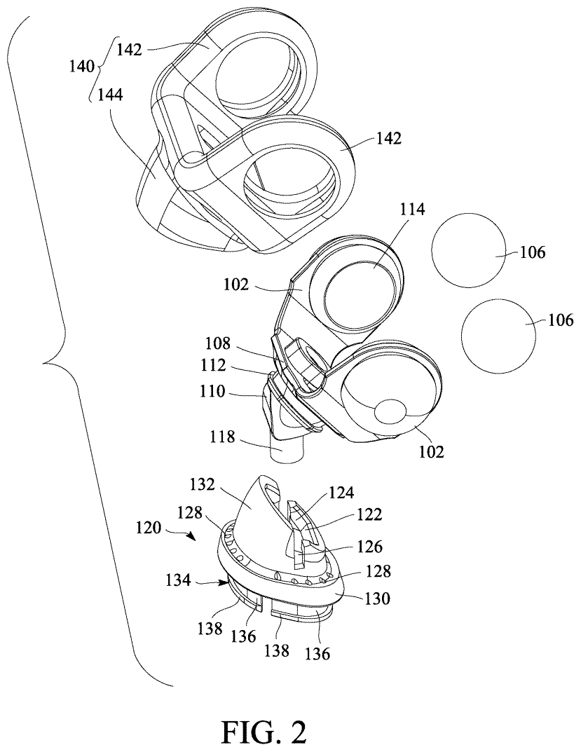

Referring to FIGS. 1A, 1B, and 2, in an embodiment of the present invention, a therapeutic massager is provided, which primarily comprises a handle 200 and a functional head 100 disposed at one end of the handle. The therapeutic device is particularly suitable for facial and body use, providing targeted stimulation, improved circulation, and enhanced absorption of skincare formulations.

The functional head 100 is provided, which includes a pair of connecting arms 102 that extend forward from a bracket 108, and a defined space between them is a massage space 104. The massage space 104 is configured to accommodate portions of the face or other body regions requiring treatment. Each connecting arm 102 is provided with a pair of rotating massage elements 106, such as a roller ball, positioned on the inner side of the connecting arm 102 facing the massage space. The rotating massage elements 106 are designed to smoothly glide along the skin surface, reducing friction and delivering consistent contact pressure during massage.

Each rotating massage element 106 is strategically mounted on the inner surface of the respective connecting arm 102 facing the other arm, such that when the user moves the device, the rotating massage element 106 rolls or glides over the skin to provide continuous, smooth stimulation. In an embodiment, the rotating massage element 106 is formed as a ball bearing rotatably housed within a mounting groove 114 formed on each connecting arm 102. The diameter of the ball bearing slightly exceeds the opening of the mounting groove 114, allowing partial projection from the surface of the connecting arm 102 while preventing it from dislodging. This design ensures that, when the ball bearing comes into contact with the skin, it can rotate freely within the mounting groove 114 as the functional head 100 is moved, thereby minimizing friction and avoiding skin irritation. The rotational movement also enhances the massage effect by stimulating local microcirculation and promoting relaxation. In an embodiment, the rotating massage element 106 may take the form of rollers, textured protrusions, or composite structures that combine different surface geometries for specialized massage sensations.

At least one of the connecting arms 102 is elastically deformable, enabling it to flex outward or inward in response to the applied pressure while massaging. This elastic deformation allows the size of the massage space 104 to vary according to the user's facial structure or the level of pressure applied during operation, ensuring a gentle yet firm grip and adaptable contact with different skin surfaces. The elastic deformation allows the spacing between the connecting arms to expand, contract, or self-adjust in response to external pressure and the geometry of the treatment area. The flexibility also enhances the effectiveness of the massage and ensures that the roller balls maintain even contact during use.

When the device is pressed against regions of varying width, such as the bridge of the nose, the jawline, or the cheekbones, the deformable connecting arm flexes outward or inward. This flexure occurs while the opposite connecting arm, or both arms simultaneously, remain biased toward their original positions. As a result, both connecting arms maintain stable contact with the skin, enabling uniform distribution of pressure and consistent engagement of the stimulation elements (e.g., rollers, massage elements, or light output).

In certain embodiments, the flexible portion covering the connecting arms includes an elastomeric material that supports controlled bending of the internal arm structure. The elastic properties of this covering further assist the arms in returning to their neutral spacing once external force is removed, thereby preserving the overall geometry of the functional head while permitting dynamic adjustment during use.

The elastic behavior ensures that the therapeutic device accommodates users having different facial or body profiles without requiring manual adjustment of the head structure. Even when used on an area that widens or narrows during movement across the face, the connecting arms deform in a manner that preserves contact, enhances treatment uniformity, and reduces the likelihood of gaps or abrupt loss of engagement between the device and the skin.

In some implementations, the arms may incorporate spring-like metal cores, flexible polymer ribs, or living hinges that enhance bending capability and allow controlled deformation within a predetermined range. These structures ensure that the arms remain resilient over repeated cycles of use.

The functional head 100 further includes a flexible cover 140 composed of a first covering portion 142 and a second covering portion 144. The first covering portion 142 overlays the connecting arms 102 and is made from a soft and biocompatible material such as silicone or thermoplastic elastomer (TPE). The first covering part 142 envelops the connecting arms 102 to provide a soft outer surface. The first covering portion 142 enhances user comfort, prevents direct contact between the rigid internal frame and the skin, and improves the overall durability and safety of the device.

Beneath the flexible cover 140, the connecting arms 102 are supported by the bracket 108 that connects them to a connecting seat 120. The connecting seat 120 joins the functional head 100 with the handle 200, ensuring structural stability. The bracket 108 and the connecting seat 120 are securely engaged through a plug-in connection. Specifically, the bracket 108 is provided with a plug-in part 110, while the connecting seat 120 includes a plug hole 122. The plug-in part 110 is inserted into the plug hole 122 to form a tight fit, providing a robust mechanical connection. When the plug-in part 110 is inserted into the plug hole 122, a locking protrusion 124 snaps into a slot 112, thereby securing the two parts together.

In an embodiment, the connecting seat 120 includes an annular wall 130, which is provided with a first annular wall 132 at one end and a second annular wall 134 at the opposite end. The first annular wall 132 and the second annular wall 134 are arranged coaxially around the annular wall 130. The first annular wall 132 is configured with one or more notches 126 that penetrate its wall structure to facilitate deformation during assembly and improve bonding with the flexible cover 140. The second annular wall 134 includes a pair of spaced-apart arc-shaped walls 136 extending circumferentially, each having multiple locking protrusions 138 on their outer surfaces for engagement with the assembly hole 218.

To further strengthen this junction, the end of the connecting seat 120 is provided with a notch 126 that penetrates through both the inner and outer walls of the first covering portion 142. A portion of the flexible cover 140 is embedded into the notch 126, thereby locking the flexible cover 140 in place and preventing detachment. Additionally, the connecting seat 120 may include multiple locking holes 128 distributed circumferentially around the joint area on the annular wall 130. During the molding process, portions of the flexible material flow into these locking holes 128 and solidify, forming a secure mechanical interlock between the flexible cover 140 and the rigid bracket 108.

In an embodiment, the therapeutic device is specifically configured to provide targeted treatment to the nose region through a combination of mechanical massage, phototherapy, and optional stimulation therapies such as vibration, microcurrent, thermal, or magnetic therapy. The functional head 100 of the device comprises a pair of connecting arms 102 defining the massage space 104 therebetween. Each connecting arm 102 is provided with the rotating massage element 106 on its inner surface facing the opposite arm, thereby allowing both the rotating massage elements 106 to come into gentle contact with the sides of the user's nose when the device is applied.

During operation, the user positions the functional head 100 such that the bridge and wings of the nose fit within the massage space 104. The elastic nature of the connecting arms 102 enables them to deform slightly outward to accommodate different nose shapes and widths, ensuring even and comfortable pressure on both sides. When the device is moved along the length of the nose or around its contours, the rotating massage elements 106 roll smoothly across the skin, stimulating local blood flow, relieving muscle tension, and assisting in the absorption of cosmetic products.

In an embodiment, one or more stimulation elements can be disposed within the functional head 100 to provide various forms of therapeutic treatment. These stimulation elements may include, but are not limited to, phototherapy elements, microcurrent electrodes, vibration motors, magnetic elements, heating and cooling elements, ultrasonic wave therapy elements, etc. The integration of such multifunctional stimulation elements enables the device to deliver comprehensive skincare and therapeutic benefits, including enhanced blood circulation, muscle relaxation, improved lymphatic drainage, deep penetration of cosmetic ingredients, and rejuvenation of skin tissues. Each stimulation element can be individually or jointly controlled through a main control board 146 to operate in different modes or intensities, allowing the user to customize the therapy according to specific needs and treatment goals.

The functional head 100 integrates a stimulation element such as a phototherapy lamp 148 positioned centrally between the two connecting arms 102. The phototherapy lamp 148 is configured to emit therapeutic light directly into the massage space 104, irradiating the skin area being massaged. The phototherapy lamp 148 may emit red, blue, infrared, or ultraviolet light, or combinations thereof, depending on the treatment mode selected by the user. These wavelengths may be used for skin rejuvenation, anti-inflammatory therapy, collagen stimulation, and improved microcirculation. The phototherapy lamp 148 is electrically connected to the main control board 146 located within the handle 200, which regulates its intensity, duration, and mode of operation.

In an embodiment, the phototherapy lamp 148 is aligned with a light-transmitting portion of the functional head 100 or a transparent compress element, allowing therapeutic light to pass through these sections for combined light and contact-based therapy. This integration of optical and mechanical functions enhances the device's effectiveness by delivering light energy directly to the treated tissue while simultaneously massaging the area.

In an embodiment, the functional head 100 further includes a stimulation element, such as a cooling or a heating compress element 156, located centrally between the connecting arms 102 and in line with the phototherapy lamp 148. The compress element 156 can be fixedly mounted or rotatably supported. When rotatable, the compress element 156 can be spherical and seated within a rolling groove formed on the bracket 108, allowing smooth and uniform contact with the skin. The compress element 156 may be made of rock (such as jade or marble), ceramic, or metal, which provides good thermal conductivity and enables temperature-based therapy such as cooling or warming effects.

If the compress element is made of transparent or translucent material, the phototherapy lamp 148 can emit light through it, achieving combined phototherapy and temperature therapy simultaneously. Alternatively, a light-transmitting hole may be formed in the center of the compress to allow the lamp's light to pass through while retaining the compress's cooling function around its edges.

In an embodiment, the stimulation element can be microcurrent electrodes. The rotating massage elements 106 can be configured not only as mechanical rollers but also as electrically conductive components capable of delivering microcurrent stimulation to the skin. Each rotating massage element 106 can be formed from or coated with a conductive material and electrically connected to the main control board 146 through internal conductive pathways within the connecting arms 102. When the device is powered, a controlled microcurrent flows through the rotating massage elements 106, forming a closed circuit with the user's skin. This gentle current stimulates facial muscles, enhances blood circulation, and promotes cellular regeneration. The combination of rolling motion and microcurrent therapy allows for simultaneous mechanical and bioelectrical stimulation, providing deeper tissue engagement and improving the skin's tone, elasticity, and absorption of cosmetic formulations during use.

In an embodiment, the stimulation element can be one or more magnetic elements embedded within the connecting arms 102 or the rotating massage elements 106 to provide magneto-therapy. These magnetic elements generate a mild magnetic field that enhances blood circulation, promotes relaxation, and supports skin regeneration. The magnets may be permanent or electromagnets controlled by the main control board 146, enabling combination therapy with light, vibration, or microcurrent stimulation for improved therapeutic results.

Referring to FIG. 2, in another configuration of the therapeutic device, the functional head 100 includes a liquid outlet port 116 positioned between the two connecting arms 102. The port is in fluid communication with a liquid channel 212 that extends through the handle 200. The liquid may originate from a detachable storage container or directly from a cosmetic container attached to the handle. A roller ball applicator may be mounted over the outlet port to control the flow of liquid and to evenly spread skincare or therapeutic formulations onto the skin during massage.

Referring to FIGS. 1A, 1B, and 2, in an embodiment, an additional rotating massage element may be disposed directly on the liquid outlet port to facilitate the application of liquid. The rotating massage element serves as both a massage element and a liquid applicator, allowing the dispensed liquid to be evenly distributed across the skin surface as it rolls. The rotation of the additional rotating massage element enhances the absorption of the cosmetic or therapeutic liquid while simultaneously delivering gentle massage stimulation, thereby improving the overall effectiveness and user experience of the device.

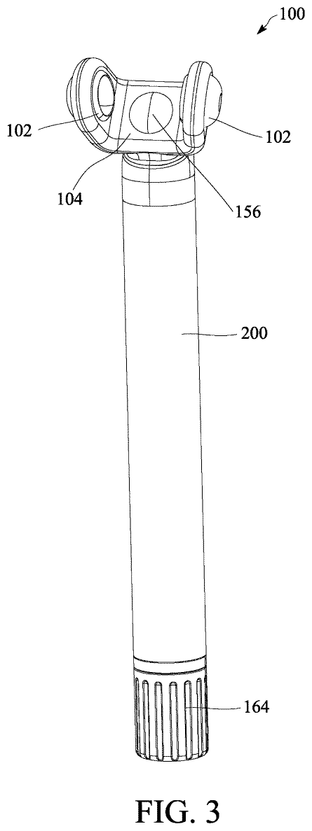

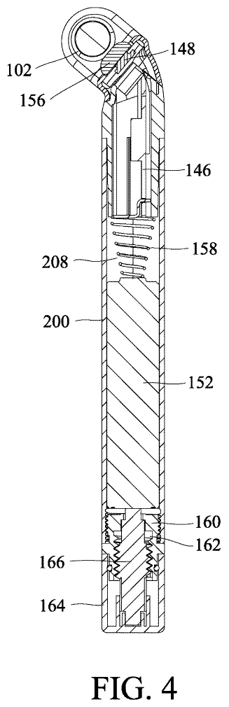

In certain embodiments, as illustrated in FIGS. 3 to 5, the therapeutic device (or massager) includes the compress element 156 disposed on the functional head 100 and positioned between the pair of connecting arms 102. The compress element 156 is designed to provide a soothing thermal effect, like a hot or cooling sensation, to the skin surface during operation. The compress element 156 may be formed from any suitable thermally conductive material, such as rock (for example, marble or jade), ceramic, or metal. When the device is applied to the skin, the compress element 156 delivers a gentle cooling sensation, thereby providing a calming effect and enhancing user comfort during treatment.

In one configuration, the compress element 156 is fixed relative to the functional head 100. In this arrangement, the compress element 156 is securely mounted between the connecting arms 102 and remains stationary during use, ensuring stable contact with the skin and simplifying the assembly structure. In an alternative configuration, the compress element 156 is spherical and rotatably mounted within the functional head 100. Specifically, the bracket 108 of the functional head 100 is provided with a rolling groove in which the compress element 156 is partially seated. This structure allows the spherical compress to rotate smoothly as it glides across the user's skin, thereby maintaining uniform surface contact and improving the cooling efficiency.

In another embodiment, the compress element 156 includes a light-transmitting portion to enable combination phototherapy. The device further comprises the phototherapy lamp 148 disposed within the functional head 100 and aligned with the light-transmitting portion of the compress. The phototherapy lamp 148 is electrically connected to the main control board 146 located within the handle 200 and configured to emit light of one or more specific wavelengths to achieve various therapeutic effects. The emitted light may include, but is not limited to, white light, red light, blue light, ultraviolet light, or infrared light, each selected based on the intended skin treatment.

The light-transmitting portion of the compress element 156 may be realized in several ways. In one embodiment, it may be formed as a light-transmitting hole through the compress element. In another embodiment, the compress element 156 can be fabricated from a transparent or translucent material, such as clear ceramic, glass, or crystal, thereby allowing therapeutic light to pass through the compress to the skin. In yet another embodiment, the compress element 156 includes a central aperture aligned with the phototherapy lamp 148, enabling direct passage of light while retaining the thermal effect around the periphery.

In a preferred configuration, the bracket 108 of the functional head 100 includes a receiving groove, within which the phototherapy lamp 148 is installed. The compress element 156 is positioned to cover this groove, creating a compact and integrated structure that is both functionally efficient and easy to assemble.

The handle 200 of the therapeutic device is generally elongated and ergonomically contoured for comfortable gripping by the user. In one embodiment, the handle 200 has a cylindrical form, facilitating ease of handling during application.

In another embodiment, the handle 200 includes an internal mounting cavity 208 that accommodates a battery 152, a conductive spring 158, a conductive element 160, and a knob 164 to run the drive mechanism. The battery 152 is slidably positioned within the internal mounting cavity 208 and is arranged to abut the conductive spring 158 at one end and the conductive element 160 at the other. The battery 152 includes positive and negative terminals, with one terminal in contact with the conductive element 160 and the opposite terminal electrically connected to the main control board 146. The conductive element 160 is also electrically connected to the main control board 146, thereby forming a complete electrical pathway.

The drive mechanism is movably mounted within the handle 200, on the side of the battery 152 opposite the conductive spring 158. The mechanism is configured to shift between a first state and a second state to control electrical connectivity. In the first state, the drive mechanism presses against the battery 152, maintaining a separation between the battery terminal and the conductive element 160, thus breaking the circuit and keeping the device powered off. In the second state, the drive mechanism is retracted, allowing the battery terminal to contact the conductive element 160, thereby completing the electrical circuit between the battery 152 and the main control board 146, and powering the device ON.

In some embodiments, the conductive element 160 is provided with a through hole 162 having internal threads. The drive mechanism includes the knob 164 and an insulating element 166 fixed within the knob 164. The knob 164 is rotatably mounted at the base of the handle 200. The insulating element 166 has external threads configured to mate with the internal threads of the through hole 162, allowing it to move axially when the knob is rotated. In the first position, the insulating element 166 extends outward from the through hole 162 to contact the battery 152, thereby spacing the battery from the conductive element 160 and disconnecting the circuit. In the second position, as the knob is rotated, the insulating element 166 retracts into the through hole 162, creating a gap between the insulating element and the battery, thereby allowing the battery terminal to contact the conductive element 160 and complete the circuit.

The handle 200 may be constructed from a conductive material, such as metal, to facilitate efficient current flow. The conductive element 160 remains in constant contact with the conductive handle surface. The conductive spring 158, formed from conductive metal, is electrically connected at one end to the main control board 146. Accordingly, when the conductive element 160 is in electrical contact with the battery 152, a closed conductive loop is formed among the battery 152, the conductive element 160, the handle 200, the main control board 146, and the conductive spring 158, energizing the entire system.

This configuration provides a simple and reliable mechanical control mechanism that enables users to conveniently power the device ON or OFF by rotating the knob 164. The integrated arrangement of the compress element 156, phototherapy lamp 148, and vibration motor 150 enables the therapeutic device to perform combined phototherapy, temperature-based therapy, and vibration massage simultaneously, delivering an enhanced and efficient skincare and relaxation experience in a single compact structure.

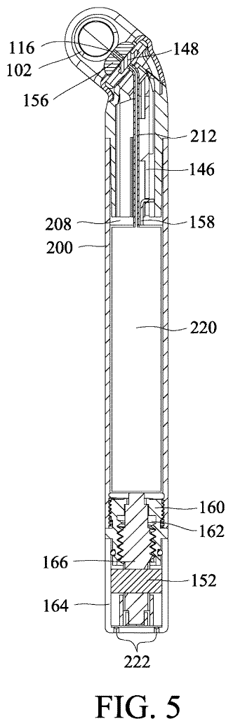

Referring to FIG. 5, in another embodiment, an alternate configuration of the therapeutic device is provided. The therapeutic device incorporates a modified power supply in which the battery 152 can be integrated within the knob 164, while the conductive pathway is disposed along the wall of the handle 200. The conductive spring 158 can be provided as a pair of elongated conductive metal strips that serve as the primary conduction pathway between the functional head 100 and the battery 152 integrated into the knob 164.

The conductive metal strip extends longitudinally from the circuitry of the functional head 100 toward the base of the handle 200, terminating at a contact region positioned adjacent the battery 152 in the knob 164. The conductive metal strip is embedded within or along the inner wall of the handle 200, fully insulated and isolated from the liquid contained within the liquid container 220. The conductive metal strips do not protrude into the liquid container 220 nor appear externally on the surface of the handle 200, thereby ensuring electrical safety, preventing corrosion, and maintaining a clean internal layout.

When the knob 164 is rotated into the ON position, the battery 152 terminal is brought into physical and electrical contact with the end of the conductive metal strip extending from the functional head 100. This rotational engagement completes the electrical circuit and supplies power to the main control board 146, stimulation element, phototherapy lamp 148, vibration motor, compress element 156, and additional elements located within the functional head 100.

Furthermore, the conductive metal strip's insulated placement within the wall of the handle 200 ensures reliable electrical conduction without risk of liquid ingress, short-circuiting, or interference with the stored cosmetic or therapeutic fluids.

In addition to the internal battery 152, the therapeutic device further includes an external electrical terminal 222 disposed at the base of the handle 200. The external electrical terminal 222 is configured to receive an auxiliary, additional, or external battery when the battery 152 of the therapeutic device is depleted. When an external battery is connected via the external electrical terminal 222, the external battery provides direct current to the therapeutic device, thereby enabling continuous operation without waiting for the battery 152 to recharge. The electrical system is designed such that the external battery seamlessly integrates with the therapeutic device's circuitry, supplying sufficient power to operate all of the components of the therapeutic device.

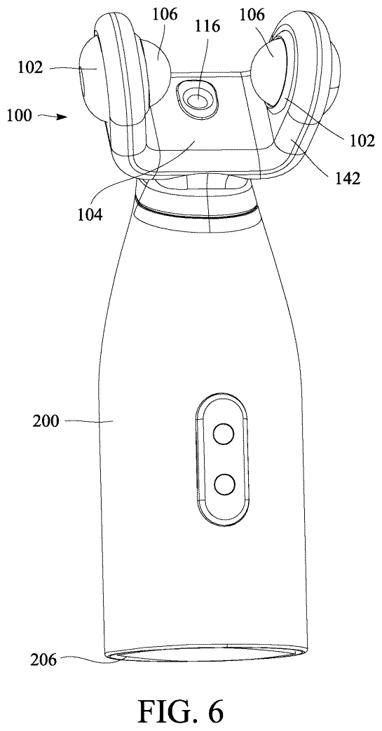

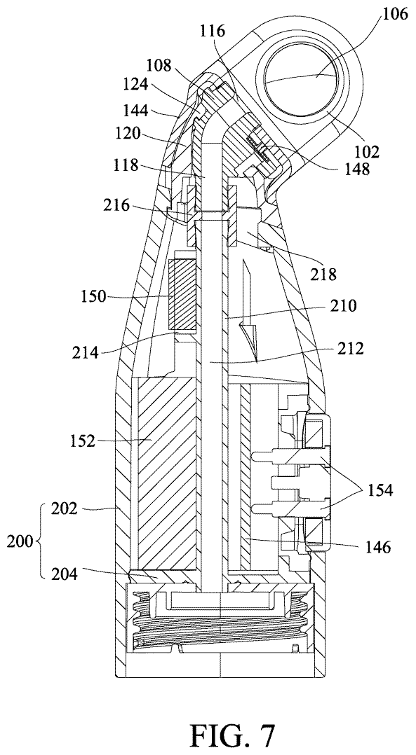

In an embodiment, referring to FIGS. 1B, 6, and 7, another configuration of the therapeutic device is disclosed. The therapeutic device comprises the handle 200 and the functional head 100 disposed at one end of the handle 200. The handle 200 serves as the main body of the device and houses the internal electrical and fluid components, while the functional head 100 is designed to deliver various therapeutic functions such as massage, phototherapy, and liquid application. The device is specifically designed to provide therapy and to apply cosmetic liquid to the nasal region, while also being suitable for application on other curved areas of the body, such as the cheekbones, eyebrows, jawline, chin, wrists, arms, forehead, and legs.

The functional head 100 includes two connecting arms 102 spaced apart to form the massage space 104 between them. Each connecting arm 102 carries the rolling massage element 106, which in this embodiment is shown as a spherical roller ball capable of rotating to provide smooth, uniform massage over the skin surface. The outer surface of each connecting arm 102 is covered by the first covering portion 142, which ensures soft contact with the skin and enhances user comfort during operation.

Positioned between the two connecting arms 102 is the liquid outlet port 116 that serves as an application or dispensing port. The liquid outlet port 116 allows cosmetic or therapeutic liquid to flow outward during use. The liquid outlet port 116 is connected to a connecting tube 118 extending downward through the bracket 108 and into a liquid guide tube 210 within the mounting bracket 204 housed inside the handle 200. Together, the connecting tube 118 and the liquid guide tube 210 define the liquid channel 212 that enables smooth passage of the liquid from the internal or external reservoir to the liquid outlet port 116.

In an embodiment, the handle 200 can include an integrated container located within the handle 200 itself. This configuration allows the liquid to be stored directly inside the handle 200, minimizing external components and providing a compact design. The liquid stored inside the integrated container travels upward through the liquid channel 212 when the device is operated, reaching the liquid outlet port 116 for direct application to the skin.

In an embodiment, in another configuration, the therapeutic device can be provided with a base portion 206. The therapeutic device can be operated with a detachable liquid container mounted at the base portion 206 of the handle 200. This container can be removably connected via a threaded or snap-fit interface, allowing users to conveniently refill, replace, or clean the container as required. When attached, the container aligns with the inlet of the liquid channel 212, establishing fluid communication between the container and the liquid outlet port 116. This versatility enables the device to function with both integrated and detachable storage options, depending on user preference or application needs.

The handle 200 further accommodates the main control board 146 that regulates electrical operation, a vibration motor 150 that transmits oscillation to the functional head 100 for mechanical massage, and the battery 152 serving as the power source. The device also includes a charging component 154, which can be a wireless coil or direct charging interface, enabling convenient recharging of the internal battery.

The phototherapy lamp 148 is embedded within the functional head 100 and positioned near the connecting arms 102 to emit light between them. This arrangement allows the user to simultaneously receive light therapy and mechanical massage during operation. The phototherapy lamp 148 may emit light in various wavelengths, such as red, blue, or infrared, providing targeted skincare benefits such as collagen stimulation, acne reduction, or improved circulation.

In an embodiment, the therapeutic device comprises an outer shell 202 forming the main housing of the handle 200, enclosing and protecting the internal electronic and mechanical components. Within the outer shell 202 is a mounting bracket 204 securely fixed to support and organize key elements such as the battery 152, main control board 146, vibration motor 150, and liquid guide tube 210. The mounting bracket 204 provides structural stability, ensuring precise alignment of internal parts while maintaining a compact form factor. It also facilitates efficient assembly and maintenance by allowing individual components to be mounted or replaced with ease. The cooperative arrangement of the outer shell 202 and the mounting bracket 204 enhances durability, prevents internal component displacement during operation, and contributes to the ergonomic and reliable design of the therapeutic device.

To ensure a sealed internal structure, the handle 200 further includes a sealing sleeve 216 connecting the liquid guide tube 210 to the connecting tube 118, thereby preventing leakage during operation. The mounting bracket 204 within the handle also includes a motor slot 214 housing the vibration motor 150, strategically positioned close to the functional head 100 to maximize the transmission of vibratory energy to the connecting arms 102 and the rotating massage elements 106.

In an embodiment, the handle 200 is provided with an assembly hole 218 configured to receive and secure the connecting seat 120 of the functional head 100. The assembly hole 218 allows the connecting seat 120, which includes the arc-shaped walls 136 and the locking protrusions 138, to be inserted and locked into position, forming a stable mechanical and electrical connection between the handle and the functional head 100. During assembly, the arc-shaped walls 136 elastically deform to allow smooth insertion into the assembly hole 218, after which the locking protrusions 138 engage with the inner wall of the hole, ensuring a firm fit.

When the device is in operation, liquid is guided either from the integrated or detachable container through the liquid channel 212 and exits through the liquid outlet port 116 located between the connecting arms 102. The user can apply gentle pressure to the skin, allowing the rotating massage elements 106 to rotate smoothly while the liquid is dispensed evenly across the surface. The combination of rolling massage, light therapy, and controlled liquid release enhances absorption of skincare products and provides a relaxing therapeutic experience.

This integrated configuration of mechanical, optical, and fluidic components in a compact design enables the multifunctional therapeutic device to deliver multiple skin and health benefits, combining convenience, versatility, and effectiveness within a single handheld structure.

For multifunctional performance, the device further incorporates one or more stimulation elements configured to provide phototherapy, microcurrent, vibration, magneto-therapy, heating therapy, cooling therapy, ultrasonic wave therapy, etc. Moreover, the vibration motor 150 is positioned within the handle 200, adjacent to the functional head 100. The vibration motor 150 is electrically connected to the main control board 146 and configured to deliver controlled vibration to the functional head 100. During operation, vibration energy is transmitted through the connecting arms 102 to the rotating massage elements 106, amplifying the massage effect and promoting relaxation of facial muscles.

The device is powered by the battery 152 contained within the handle 200, and may also include the charging component 154 configured for wired or wireless charging. The handle 200 features the internal mounting bracket 204 that supports the battery 152, main control board 146, and the liquid guide tube 210 incorporating the liquid channel 212. The bracket 108 of the functional head 100 is provided with a connecting tube 118 communicating with the liquid outlet port 116, which connects to the sealing sleeve 216 that links the head assembly to the liquid guide tube 210. This arrangement ensures an airtight liquid transfer pathway while maintaining the compactness of internal components. The motor slot 214 within the mounting bracket 204 accommodates the vibration motor 150, allowing efficient vibration transmission and a compact internal configuration.

In an embodiment, the rotating massage elements may be configured to function as liquid applicators. A liquid dispensing port can be disposed beneath each rotating massage element, allowing skincare or therapeutic liquid to be released directly onto the skin during massage. When the device is operated, the liquid from the integrated or detachable container flows through an internal liquid channel and exits through the port positioned below the rotating element. As the rotating massage element rolls over the skin, the liquid is evenly spread across the treated area, enabling simultaneous massage and topical application. This configuration ensures efficient absorption of the liquid while enhancing the overall therapeutic effect through the combined action of rolling stimulation and targeted liquid delivery.

In an embodiment of the present invention, a method for using a therapeutic device to provide one or more therapies to a user is provided. In use, the therapeutic device is operated by holding the handle and placing the functional head against a target surface of the human body, such as the cheeks, chin, forehead, nose, jawline, or other curved regions.

The functional head includes a pair of connecting arms defining a massage space. Each connecting arm may carry one or more stimulation elements such as a phototherapy lamp, vibration element, heating or cooling component, or a massage roller ball. When the functional head is applied to the skin, the stimulation elements are activated to deliver therapeutic effects, including phototherapy, vibration massage, temperature modulation, or microcurrent stimulation.

The phototherapy lamp may emit light of one or more wavelengths selected from red, blue, infrared, or ultraviolet light. Red and infrared wavelengths promote collagen production and skin rejuvenation, while blue light provides antibacterial action for acne-prone areas.

In some embodiments, the connecting arms are elastically deformable such that the spacing between them varies according to the user's facial contour or the applied pressure. This allows the functional head to maintain uniform contact over curved regions of the body while delivering the desired stimulation.

At least one of the connecting arms 102 is elastically deformable, enabling it to flex outward or inward in response to the applied pressure while massaging. This elastic deformation allows the size of the massage space 104 to vary according to the user's facial structure or the level of pressure applied during operation, ensuring a gentle yet firm grip and adaptable contact with different skin surfaces. The elastic deformation allows the spacing between the connecting arms to expand, contract, or self-adjust in response to external pressure and the geometry of the treatment area. The flexibility also enhances the effectiveness of the massage and ensures that the roller balls maintain even contact during use.

When the device is pressed against regions of varying width, such as the bridge of the nose, the jawline, or the cheekbones, the deformable connecting arm flexes outward or inward. This flexure occurs while the opposite connecting arm, or both arms simultaneously, remain biased toward their original positions. As a result, both connecting arms maintain stable contact with the skin, enabling uniform distribution of pressure and consistent engagement of the stimulation elements (e.g., rollers, massage elements, or light output).

In certain embodiments, the flexible portion covering the connecting arms includes an elastomeric material that supports controlled bending of the internal arm structure. The elastic properties of this covering further assist the arms in returning to their neutral spacing once external force is removed, thereby preserving the overall geometry of the functional head while permitting dynamic adjustment during use.

The elastic behavior ensures that the therapeutic device accommodates users having different facial or body profiles without requiring manual adjustment of the head structure. Even when used on an area that widens or narrows during movement across the face, the connecting arms deform in a manner that preserves contact, enhances treatment uniformity, and reduces the likelihood of gaps or abrupt loss of engagement between the device and the skin.

In some implementations, the arms may incorporate spring-like metal cores, flexible polymer ribs, or living hinges that enhance bending capability and allow controlled deformation within a predetermined range. These structures ensure that the arms remain resilient over repeated cycles of use.

During assembly, the functional head may be fixed to the handle through a mechanical coupling between a support and a connecting seat. The support may include a plug-in part that inserts into a corresponding plug hole of the connecting seat. The connecting seat can include one or more notches and locking holes into which portions of a flexible covering part are embedded, ensuring a secure and vibration-resistant connection between the handle and the functional head. This coupling structure enables the stable operation of the stimulation elements during therapeutic use.

In another embodiment, the therapeutic device is configured to apply a cosmetic or therapeutic liquid while simultaneously performing stimulation. The device includes a liquid channel extending through the handle and communicating with an outlet port provided on the functional head. The liquid channel is in fluid communication with a detachable liquid storage container mounted to the handle or functional head.

During use, the user positions the functional head against the body surface and actuates the device to dispense liquid from the storage container through the liquid channel. The liquid exits the outlet port positioned between the connecting arms and spreads onto the user's skin.

In some embodiments, a roller ball or similar movable dispensing element is positioned at the outlet port. As the roller ball moves over the skin, it evenly transfers the liquid to the surface while simultaneously performing mechanical massage.

The dispensing of liquid may be coordinated with activation of the stimulation elements, such that light, vibration, or temperature therapy is applied in combination with topical treatment. This integrated function enhances absorption of the liquid formulation and improves the overall therapeutic effect. After use, the liquid storage container can be easily detached from the handle for refilling or replacement.

The device further includes a knob-controlled power mechanism for selectively connecting or disconnecting the internal electrical circuit. The knob is positioned at the base of the handle and is manually rotatable between OFF and ON positions. When the user rotates the knob, an insulating element housed inside the handle is retracted, permitting the terminal of the battery to contact a conductive element. This contact completes an electrical circuit extending through the conductive element, spring, handle, and main control board, thereby energizing the phototherapy lamp, vibration motor, or other stimulation components in the functional head.

Rotating the knob in the opposite direction causes the insulating element to return to its initial position, separating the battery terminal from the conductive element and interrupting the circuit to turn the device OFF. The knob-controlled mechanism provides a simple, tactile means of power control without requiring electronic switches, making the design more robust and user-friendly.

In certain embodiments, the main control board regulates the output intensity or mode of the stimulation elements according to user settings. The handle may further include a wired charging port or a wireless charging coil for replenishing the battery. This arrangement enables continuous operation and convenient charging while maintaining a compact, sealed structure.

The present invention provides a therapeutic device that combines massage, phototherapy, vibration, microcurrent, temperature regulation, and liquid application within a single compact and ergonomic structure. Its innovative configuration of elastic connecting arms, rotatable massage elements, and integrated light and vibration systems enables precise, comfortable, and effective treatment across various facial and body regions. The incorporation of a knob-controlled power mechanism ensures user-friendly operation, while the detachable liquid delivery system enhances convenience and hygiene. This invention offers significant advantages in the personal care, beauty, and healthcare industries, addressing the increasing demand for multifunctional and portable therapeutic tools. The versatility of the device allows it to be adapted for home use, professional spa treatments, and clinical skincare applications, promoting relaxation, improved circulation, and enhanced absorption of topical formulations. Thus, the invention presents a practical, efficient, and commercially valuable advancement in the field of therapeutic and cosmetic devices.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A therapeutic device, comprising:
a handle; and
a functional head disposed at one end of the handle, the functional head comprises a pair of connecting arms spaced apart to define a massage space;
a phototherapy element disposed in the massage space for emitting therapeutic light between the connecting arms;
at least one stimulation element disposed in the functional head; and
wherein the stimulation element is configured to deliver one or more therapies to a user.

2. The therapeutic device of claim 1, wherein the connecting arms are elastically deformable to vary a width of the massage space.

3. The therapeutic device of claim 1, wherein each connecting arm includes a rotatable massage element configured to provide a massage therapy to the user.

4. The therapeutic device of claim 1, wherein at least one stimulation element comprises one of a heating element, a cooling element, a Peltier component, a magnetotherapy element, a vibration element, or a micro-current element.

5. The therapeutic device of claim 1, wherein the functional head further comprises a flexible cover enclosing at least a part of the connecting arms.

6. The therapeutic device of claim 5, wherein the functional head further comprises a support and a connecting seat, the support connecting the pair of connecting arms, and the connecting seat being fixed to the handle.

7. The therapeutic device of claim 6, wherein the support includes a plug-in part, and the connecting seat includes a plug hole, the plug-in part being inserted into the plug hole to couple the support and the connecting seat.

8. The therapeutic device of claim 6, wherein an end of the connecting seat includes a notch penetrating the inner and outer sides of the connecting seat, and a first covering portion of the flexible cover is embedded within the notch.

9. The therapeutic device of claim 6, wherein the connecting seat includes a plurality of locking holes, and a second covering portion of the flexible cover is embedded within the locking holes to secure the functional head.

10. The therapeutic device of claim 1, wherein the functional head is inclined relative to the handle at an obtuse angle to facilitate contact with curved facial regions, including nose, jawline, cheekbones, and forehead.

11. A therapeutic device, comprising:
a handle;
a functional head disposed at one end of the handle, the functional head having a liquid outlet port positioned between a pair of connecting arms;
one or more stimulation elements disposed in the functional head for providing one or therapies to a user;
a liquid channel extending through the handle and communicating with the liquid outlet port; and
a liquid storage container detachably coupled to the handle and in fluid communication with the liquid channel,
wherein the liquid storage container is configured to deliver cosmetic or therapeutic liquid to the functional head.

12. The therapeutic device of claim 11, wherein a roller ball is disposed at the liquid outlet port and configured to dispense liquid upon rolling contact with the skin of the user.

13. The therapeutic device of claim 11, wherein the handle includes a mounting portion for attachment to the liquid storage container.

14. The therapeutic device of claim 13, wherein the liquid channel extends from an inlet at the mounting portion to the liquid outlet port of the functional head.

15. The therapeutic device of claim 11, wherein the liquid storage container is removably attachable to enable replenishment or cleaning.

16. The therapeutic device of claim 11, wherein one or more stimulation elements are selected from a phototherapy lamp or vibration module to provide combined liquid application and therapy.

17. A therapeutic device, comprising:
a handle configured to house a battery and a main control board;
a functional head disposed at one end of the handle and including at least one stimulation element electrically connected to the main control board; and
a knob-controlled power mechanism comprising a knob, disposed at a base of the handle, wherein rotation of the knob retracts an insulating element to allow a terminal of the battery to contact a conductive element, thereby completing an electrical circuit through the conductive element, a spring, and the main control board to power the stimulation element.

18. The therapeutic device of claim 17, wherein the knob is manually rotatable between ON and OFF positions.

19. The therapeutic device of claim 18, wherein the spring biases the insulating element toward the terminal of the battery in the OFF position to prevent electrical contact.

20. The therapeutic device of claim 17, wherein the main control board is configured to regulate the operation of the stimulation element according to user-selected modes.
